(19) United States
(12) Patent Application Publication
(10) Pub. No.: US 2024/0355451 A1
(43) Pub. Date: Oct. 24, 2024

(54) METHODS FOR IMPROVING GUT MICROBIOTA DIVERSITY

(71) Applicant: SOCIETE DES PRODUITS NESTLE S.A., Vevey (CH)

(72) Inventors: CAROLINE LE ROY, Lausanne (CH); SHAILLAY KUMAR DOGRA, Epalinges (CH)

(21) Appl. No.: 18/684,723

(22) PCT Filed: Aug. 22, 2022
(86) PCT No.: PCT/EP2022/073323

§ 371 Date: Feb. 19, 2024

(30) Foreign Application Priority Data
Aug. 25, 2021 (EP) 21193064.9

(51) Int. Cl. G16H 20/60

(52) U.S. Cl. CPC G16H 20/60

(57) ABSTRACT

The present invention relates to a method for determining the effects of a food product, meal, or diet on a subject's gut microbiota, wherein the method comprises determining the sum of different types of polyphenol present in the food product, meal, or diet.

### FIELD OF THE INVENTION

The present invention relates to a method for determining the effects of a food product, meal, or diet on a subject's gut microbiota, comprising determining the sum of different types of polyphenol present in the food product, meal, or diet. The present invention further relates to a dietary index for assessing the effects of a food product, meal, or diet on a subject's gut microbiota.

### BACKGROUND TO THE INVENTION

The diversity and composition of the microbial community residing in the large intestine (gut microbiome) is tightly linked to the health status of its host (Lynch & Pedersen, 2016, New England Journal of Medicine, 375(24), 2369-2379). The composition of this dynamic ecosystem is partly shaped by environmental factors including diet (Zhernakova et al., 2016, Science, 352(6285), 565-569; Falony et al., 2016, Science, 352(6285), 560-564).

Within diet, some components such as polyphenols are recognised to have greater effects on the gut microbiome (Gibson, G.R., et al., 2017. Nature reviews Gastroenterology & hepatology, 14(8), pp.491-502). Polyphenols are phytochemical produced by plants to protect them against UV light and pathogens. With over 8000 different compounds this is a large family of chemicals known to exert beneficial effects on human health (Scalbert, A., et al., 2002. Biomedicine & Pharmacotherapy, 56(6), pp.276-282).

Part of the beneficial effects of polyphenols on health is mediated by the gut microbiome (Mompeo, O., et al., 2020. Nutrients, 12(6), p.1871). Several mechanisms are at play, including: (1) promotion of the growth of beneficial bacteria; (2) modification of the gut lumen environment; (3) antimicrobial effects against pathogens; and (4) metabolization by bacteria before absorption through the gut barrier (Van Hul, M. and Cani, P.D., 2019. Current nutrition reports, 8(4), pp.307-316; Ma, J., et al., 2020. Animal Nutrition, 6(4), pp.404-409).

Although effects of individual phenolic compounds on the gut microbiome have been explored, their variety and prevalence in the human diet implies that they are never consumed as single entities but in combinations and with different levels of diversity. The effects of the diversity of polyphenol intake on the gut microbiome has not been explored and prevents full understanding of the role of polyphenols on gut microbiome composition.

### SUMMARY OF THE INVENTION

The inventors have developed a system which assesses the diversity of polyphenols in a food product, meal, or diet, to support the development and/or maintenance of a healthy gut microbiome.

The inventors have shown that the system can be used to develop a new index capturing the richness and/or diversity of polyphenols in a food product, meal, or diet. Such an index may reflect the potential of a food product, meal or diet to enhance gut microbiota diversity and the growth of beneficial taxa. The index may be used to adjust food products, meals, or diets to ensure a high diversity of polyphenol intake.

In one aspect, the present invention provides a method for determining the effects of a food product, meal, or diet on a subject's gut microbiota, wherein the method comprises determining the sum of different types of polyphenol present in the food product, meal, or diet.

In some embodiments, the method further comprises determining the amount of each different type of polyphenol present in the food product, meal, or diet. In some embodiments, the method further comprises determining the diversity of polyphenols present in the food product, meal, or diet. The diversity of polyphenols present in the food product, meal, or diet may be determined using a polyphenol diversity index.

A greater sum of different types of polyphenol present in the food product, meal, or diet provides a more diverse gut microbiota. A greater diversity of polyphenols present in the food product, meal, or diet may provide a more diverse gut microbiota. A more diverse gut microbiota may have: (i) a higher number of observed microbial taxa; and/or (ii) a higher diversity index, preferably wherein the diversity index is a phylogenetic diversity index, such as a Faith's PD index, or a Shannon index.

The different types of polyphenol may comprise flavonoids, lignans, stilbenes, and phenolic acids. The different types of polyphenol may comprise flavonols, flavanones, flavones, flavan-3-ols, anthocyanins/anthocyanidins, and isoflavones. The different types of polyphenol may comprise flavonols, flavanones, flavones, catechins, theaflavins, thearubigins, anthocyanins/anthocyanidins, and isoflavones. In some embodiments, the different types of polyphenol comprise: (−)-Epicatechin, (−)-Epicatechin 3-gallate, (−)-Epigallocatechin, (−)-Epigallocatechin 3-gallate, (+)-Catechin, (+)-Gallocatechin, Apigenin, Cyanidin, Daidzein, Delphinidin, Eriodictyol, Genistein, Glycitein, Hesperetin, Isorhamnetin, Kaempferol, Luteolin, Malvidin, Myricetin, Naringenin, Pelargonidin, Peonidin, Petunidin, Quercetin, Theaflavin, Theaflavin-3-gallate, Theaflavin-3,3′-digallate, Theaflavin-3′-gallate, and Total Thearubigins.

In some embodiments, the different types of polyphenol comprise or consist of: (−)-Epicatechin, (−)-Epicatechin 3-gallate, (−)-Epigallocatechin, (−)-Epigallocatechin 3-gallate, (+)-Catechin, (+)-Gallocatechin, Apigenin, Cyanidin, Daidzein, Delphinidin, Eriodictyol, Genistein, Glycitein, Hesperetin, Isorhamnetin, Kaempferol, Luteolin, Malvidin, Myricetin, Naringenin, Pelargonidin, Peonidin, Petunidin, Quercetin, Theaflavin, Theaflavin-3-gallate, Theaflavin-3,3′-digallate, Theaflavin-3′-gallate, Total Thearubigins, Total anthocyanidins, Total catechins, Total flavan-3-ols, Total flavanones, Total flavones, Total flavonoids, Total flavonols, and Total isoflavones.

The method may further comprise determining a dietary index using the sum of different types of polyphenol present in the food product, meal, or diet. A higher dietary index may provide a more diverse gut microbiota. In some embodiments, the dietary index comprises or consists of the sum of different types of polyphenol present in the food product, meal, or diet. In some embodiments, the method comprises determining a dietary index using the amount of each different type of polyphenol present in the food product, meal, or diet and/or the similarity in the chemical structure of the different types of polyphenol present in the food product, meal, or diet. In some embodiments, the method comprises determining a dietary index using the diversity of polyphenols present in the food product, meal, or diet, optionally wherein the diversity of polyphenols present in the food product, meal, or diet is determined using a polyphenol diversity index. In some embodiments, the dietary index comprises or consists of a polyphenol diversity index.

In another aspect, the present invention provides a dietary index for assessing the effects of a food product, meal, or diet on a subject's gut microbiota. The dietary index may be determined using a method of the invention.

In another aspect, the present provides method for providing a dietary index according to the present invention, wherein the method comprises determining the dietary index using the sum of different types of polyphenol present in a food product, meal, or diet.

In another aspect, the present invention provides a method for adjusting a food product, meal, or diet, wherein the method comprises:
(a) determining the effects of the food product, meal, or diet on a subject's gut microbiota using a method according to the present invention; and
(b) adjusting the food product, meal, or diet to provide an adjusted food product, adjusted meal, or adjusted diet with an improved effect on the subject's gut microbiota diversity.

The adjusted food product, adjusted meal, or adjusted diet may provide a greater number of different types of polyphenol than the un-adjusted food product, un-adjusted meal, or un-adjusted diet.

In another aspect, the present invention provides a method for maintaining or improving a subject's gut microbiota diversity, wherein the method comprises:
(a) determining the effects of the subject's present diet on the subject's gut microbiota using a method according to the present invention; and
(b) adjusting the subject's present diet to provide an adjusted diet with an improved effect on the subject's gut microbiota diversity.

In another aspect, the present invention provides a method for maintaining or improving a subject's gut microbiota diversity, wherein the method comprises:
(a) determining a dietary index according to the present invention for the subject's present diet; and
(b) adjusting the subject's present diet to provide an adjusted diet with an improved dietary index.

The adjusted diet may provide a greater number of different types of polyphenol than the subject's present diet. After adjusting the diet, the gut microbiota status of the subject may be healthy. The adjusted diet may increase the abundance and/or function of favourable microbial taxa and/or may decrease the abundance and/or function of unfavourable microbial taxa.

In another aspect, the present invention provides use of the sum of different types of polyphenol present in a food product, meal, or diet to assess the effects of the food product, meal, or diet on a subject's gut microbiota.

In another aspect, the present invention provides use of a polyphenol diversity index to assess the effects of the food product, meal, or diet on a subject's gut microbiota.

In another aspect, the present invention provides use of a dietary index according to the present invention for assessing the effects of a food product, meal, or diet on a subject's gut microbiota.

The methods of the present invention may be computer-implemented methods.

In another aspect, the present invention provides a computer program comprising instructions which, when the program is executed by a computer, cause the computer to carry out a method according to the present invention.

In another aspect, the present invention provides a computer program comprising instructions which, when the program is executed by a computer, cause the computer to determine a dietary index for assessing the effects of a food product, meal, or diet on a subject's gut microbiota, given the different types of polyphenol present in the food product, meal, or diet.

In another aspect, the present invention provides a computer-readable medium comprising instructions which, when executed by a computer, cause the computer to carry out a method according to the present invention.

In another aspect, the present invention provides a computer-readable medium comprising instructions which, when executed by a computer, cause the computer to determine a dietary index for assessing the effects of a food product, meal, or diet on a subject's gut microbiota, given the different types of polyphenol present in the food product, meal, or diet.

### DESCRIPTION OF DRAWINGS

FIG. 1—histogram of the distribution of the polyphenol diversity index in a population

A polyphenol diversity index was constructed based on the presence or absence of 38 polyphenols in the 259 food items present in the food frequency questionnaire (FFQ) distributed to American gut project (AGP) participants. The polyphenol diversity index was calculated as the sum of all polyphenols consumed by a participant.

### DETAILED DESCRIPTION

Various preferred features and embodiments of the present invention will now be described by way of non-limiting examples. The skilled person will understand that they can combine all features of the invention disclosed herein without departing from the scope of the invention as disclosed.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

### Method for Determining the Effects of a Food Product, Meal, or Diet

The present invention provides a method for determining the effects of a food product, meal, or diet on a subject's gut microbiota, wherein the method comprises determining the sum of different types of polyphenol present in the food product, meal, or diet.

### Polyphenols

“Polyphenols” (also known as “phenolic compounds”) are a large family of organic compounds characterized by having at least one phenol unit and includes flavonoids, lignans, stilbenes, and phenolic acids (see e.g. Manach, C., et al., 2004. The American journal of clinical nutrition, 79(5), pp.727-747). In some embodiments, the polyphenols have two or more phenol units.

Suitably, the polyphenols are dietary polyphenols. “Dietary polyphenols” may refer to polyphenols that are commonly consumed. Types of dietary polyphenol are well known in the art, for example as described in Scalbert, A., et al., 2002. Biomedicine & Pharmacotherapy, 56(6), pp.276-282. Phenol-Explorer is an online comprehensive database on polyphenol contents in foods (see Neveu, V., Perez-Jiménez, J., et al., 2010. Database, 2010, bap024; Rothwell, J.A., et al., 2012. Database, 2012, bas031; and Rothwell, J.A. et al., 2013. Database, 2013, bat070).

The “types” of polyphenols may include any polyphenol class, polyphenol sub-class, or individual polyphenol. For example, each of “Total flavonoids”, “Total flavanols”, “Total catechins”, and (+)-Catechin may be considered a “type” of polyphenol.

### Flavonoids

In some embodiments, the different types of polyphenol comprise flavonoids. In some embodiments, the different types of polyphenol comprise Total flavonoids (i.e. the flavonoids are grouped together and classified as one “type” of polyphenol). The term “flavonoid” includes natural and synthetic products and may be applied to: (1) compounds whose structural feature is based on derivatives of a phenyl-substituted propylbenzene possessing a C15 skeleton; (2) compounds having a C16 skeleton that are phenyl-substituted propylbenzene derivatives (rotenoids); and (3) flavonolignans, whose structure is based on derivatives of phenyl-substituted propylbenzene condensed with C6-C3 lignan precursors (Rauter, A.P., et al., 2018. Pure and Applied Chemistry, 90(9), pp. 1429-1486).

In some embodiments, the different types of polyphenol comprise one or more class of flavonoid. Exemplary classes of flavonoids include anthocyanins, chalcones, dihydrochalcones, dihydroflavonols, flavanols, flavanones, flavones, flavonols, and isoflavonoids. In some embodiments, the different types of polyphenol comprise one or more of: Total anthocyanins/anthocyanidins, Total chalcones, Total dihydrochalcones, Total dihydroflavonols, Total flavanols, Total flavanones, Total flavones, Total flavonols, and Total isoflavonoids.

In some embodiments, the different types of polyphenol comprise anthocyanins and/or anthocyanidins. In some embodiments, the different types of polyphenol comprise Total anthocyanins and/or anthocyanidins (i.e. the anthocyanins and/or anthocyanidins are grouped together and classified as one “type” of polyphenol). In some embodiments, the different types of polyphenol comprise one or more anthocyanin and/or anthocyanidin. “Anthocyanidins” may include oxygenated derivatives of flavylium (2-phenylchromenylium) salts. “Anthocyanins” may be derived from anthocyanidins by adding sugars. Exemplary anthocyanins/anthocyanidins include: Malvidin 3-O-(6″-p-coumaroyl-glucoside), Delphinidin 3-O-(6″-acetyl-galactoside), Cyanidin 3-O-(6″-acetyl-galactoside), Cyanidin 3-O-galactoside, Cyanidin 3-O-glucoside, Cyanidin 3-O-rutinoside, Cyanidin 3-O-sophoroside, Pelargonidin 3-O-glucoside, Cyanidin 3-O-(6″-malonyl-glucoside), Peonidin, Peonidin 3-O-glucoside, Peonidin 3-O-rutinoside, Pelargonidin 3-O-rutinoside, Pelargonidin, Cyanidin, Malvidin 3,5-O-diglucoside, Cyanidin 3-O-glucosyl-rutinoside, Pelargonidin 3-O-sophoroside, Pelargonidin 3-O-glucosyl-rutinoside, Cyanidin 3-O-(6″-succinyl-glucoside), Pelargonidin 3-O-(6″-succinyl-glucoside), Delphinidin 3-O-galactoside, Delphinidin 3-O-glucoside, Delphinidin 3-O-arabinoside, Petunidin 3-O-galactoside, Cyanidin 3-O-arabinoside, Petunidin 3-O-glucoside, Peonidin 3-O-galactoside, Petunidin 3-O-arabinoside, Malvidin 3-O-glucoside, Malvidin 3-O-arabinoside, Delphinidin 3-O-(6″-acetyl-glucoside), Petunidin 3-O-(6″-acetyl-galactoside), Peonidin 3-O-(6″-acetyl-galactoside), Cyanidin 3-O-(6″-acetyl-glucoside), Malvidin 3-O-(6″-acetyl-galactoside), Petunidin 3-O-(6″-acetyl-glucoside), Malvidin 3-O-(6″-acetyl-glucoside), Peonidin 3-O-(6″-acetyl-glucoside), Pelargonidin 3-O-arabinoside, Delphinidin 3-O-rutinoside, Cyanidin 3-O-sambubioside, Pelargonidin 3-O-(6″-malonyl-glucoside), Peonidin 3-O-(6″-p-coumaroyl-glucoside), Cyanidin 3-O-xyloside, Malvidin 3-O-galactoside, Peonidin 3-O-arabinoside, Petunidin 3-O-rutinoside, Delphinidin 3-O-xyloside, Petunidin 3-O-(6″-p-coumaroyl-glucoside), Pelargonidin 3-O-galactoside, Pelargonidin 3-O-sambubioside, Delphinidin 3-O-sambubioside, Cyanidin 3-O-xylosyl-rutinoside, Vitisin A, Delphinidin 3-O-(6″-p-coumaroyl-glucoside), Pigment A, Cyanidin 3-O-(6″-p-coumaroyl-glucoside), Cyanidin 3-O-sambubioside 5-O-glucoside, Cyanidin 3-O-(6″-caffeoyl-glucoside), Cyanidin 3,5-O-diglucoside, Pinotin A, Delphinidin 3,5-O-diglucoside, Pelargonidin 3,5-O-diglucoside, Malvidin 3-O-(6″-caffeoyl-glucoside), Cyanidin 3-O-(6″-dioxalyl-glucoside), Delphinidin 3-O-glucosyl-glucoside, Cyanidin 3-O-(6″-malonyl-3″-glucosyl-glucoside), Delphinidin 3-O-feruloyl-glucoside, Petunidin 3,5-O-diglucoside, Petunidin 3-O-rhamnoside, Cyanidin 3-O-diglucoside-5-O-glucoside, Peonidin 3-O-diglucoside-5-O-glucoside, Peonidin 3-O-(2-O-(6-O-(E)-caffeoyl-D-glucosyl)-D-glucoside)-5-O-D-glucoside, Peonidin 3-O-sophoroside, Peonidin 3-O-sambubioside, Peonidin 3-O-sambubioside-5-O-glucoside, Peonidin 3-O-xyloside, 4′-O-Methylcyanidin 3-O-D-glucoside, Cyanidin 3-O-(3″,6″-O-dimalonyl-glucoside), 4-O-Methyldelphinidin 3-O-D-glucoside, Isopeonidin 3-O-arabinoside, Isopeonidin 3-O-galactoside, Isopeonidin 3-O-glucoside, Isopeonidin 3-O-rutinoside, Isopeonidin 3-O-sambubioside, Isopeonidin 3-O-xyloside, Cyanidin 3-O-(2-O-(6-O-(E)-caffeoyl-D glucoside)-D-glucoside)-5-O-D-glucoside, and 4′-O-Methyldelphinidin 3-O-rutinoside.

In some embodiments, the different types of polyphenol comprise chalcones. In some embodiments, the different types of polyphenol comprise Total chalcones (i.e. the chalcones are grouped together and classified as one “type” of polyphenol). In some embodiments, the different types of polyphenol comprise one or more chalcone. “Chalcones” may include 1,3-Diphenylpropenone (benzylideneacetophenone), and its derivatives formed by substitution. Exemplary chalcones include butein and xanthohumol.

In some embodiments, the different types of polyphenol comprise dihydrochalcones. In some embodiments, the different types of polyphenol comprise Total dihydrochalcones (i.e. the dihydrochalcones are grouped together and classified as one “type” of polyphenol). In some embodiments, the different types of polyphenol comprise one or more dihydrochalcone. “Dihydrochalcones” may include 1,3-Diphenylpropanone, and its derivatives formed by substitution. Exemplary dihydrochalcones include: Phloretin, Phloridzin, Phloretin 2′-O-xylosyl-glucoside, 3-Hydroxyphloretin 2′-O-xylosyl-glucoside, 3-Hydroxyphloretin 2′-O-glucoside, and Phloretin 2′-O-glucuronide.

In some embodiments, the different types of polyphenol comprise dihydroflavonols. In some embodiments, the different types of polyphenol comprise Total dihydroflavonols (i.e. the dihydroflavonols are grouped together and classified as one “type” of polyphenol). In some embodiments, the different types of polyphenol comprise one or more dihydroflavonol. “Dihydroflavonols” (also known as “flavanonols”) may include flavonoids that use the 3-hydroxy-2,3-dihydro-2-phenylchromen-4-one backbone. Exemplary dihydroflavonols include: Dihydroquercetin 3-O-rhamnoside, Dihydroquercetin, and Dihydromyricetin 3-O-rhamnoside.

In some embodiments, the different types of polyphenol comprise flavanols. In some embodiments, the different types of polyphenol comprise Total flavanols (i.e. the flavanols are grouped together and classified as one “type” of polyphenol). In some embodiments, the different types of polyphenol comprise one or more flavanol. “Flavanols” (also known “flavan-3-ols”) may include derivatives of flavans that possess a 2-phenyl-3,4-dihydro-2H-chromen-3-ol skeleton. Exemplary flavanols include: (+)-Catechin, (−)-Epicatechin, (+)-Gallocatechin, (−)-Epigallocatechin, (−)-Epicatechin 3-O-gallate, (−)-Epigallocatechin 3-O-gallate, Theaflavin, Theaflavin 3-O-gallate, Theaflavin 3′-O-gallate, Theaflavin 3,3′-O-digallate, (+)-Gallocatechin 3-O-gallate, (+)-Catechin 3-O-gallate, Procyanidin dimer B1, Procyanidin dimer B2, Procyanidin dimer B3, Procyanidin dimer B4, Procyanidin dimer B5, Procyanidin dimer B7, Prodelphinidin dimer B3, Procyanidin trimer C1, Procyanidin trimer EEC, Procyanidin trimer T2, Procyanidin trimer C2, Prodelphinidin trimer GC-GC-C, Prodelphinidin trimer GC-C-C, Prodelphinidin trimer C-GC-C, (−)-Epicatechin-(2a-7)(4a-8)-epicatechin 3-O-galactoside, Cinnamtannin A2, (+)-Catechin 3-O-glucose, 3′-O-Methylepicatechin, 4′-O-Methyl-(−)-epicatechin 3′-O-glucuronide, Epicatechin 3′-O-glucuronide, 3′-O-Methylcatechin, 4′,4″-O-Dimethylepigallocatechin 3-O-gallate, 4′-O-Methylepigallocatechin, 4″-O-Methylepigallocatechin 3-O-gallate, 4′-O-Methylepicatechin, Epigallocatechin 3-O-gallate-7-O-glucoside-4″-O-glucuronide, (−)-Epigallocatechin 3-O-glucuronide, 3′-O-Methyl-(−)-epicatechin 7-O-glucuronide, Epicatechin 7-O-glucuronide, (−)-Epigallocatechin 3′-O-glucuronide, (−)-Epigallocatechin 7-O-glucuronide, 4′-O-Methyl-(−)-epigallocatechin 3′-O-glucuronide, and 4′-O-Methyl-(−)-epigallocatechin 7-O-glucuronide. In some embodiments, the different types of polyphenol comprise one or more class of flavanol. Exemplary classes of flavanols include catechins, theaflavins, and thearubigins. In some embodiments, the different types of polyphenol comprise one or more of: Total catechins, Total theaflavins, and Total thearubigins (i.e. each of the catechins, theaflavins, and/or thearubigins are separately grouped together and each classified as one “type” of polyphenol).

In some embodiments, the different types of polyphenol comprise flavanones. In some embodiments, the different types of polyphenol comprise Total flavanones (i.e. the flavanones are grouped together and classified as one “type” of polyphenol). In some embodiments, the different types of polyphenol comprise one or more flavanone. “Flavanones” may include flavonoids that have the basic 2,3-dihydroflavone structure and lacking a double bond between C2 and C3. Exemplary flavanones include: Naringenin, Eriodictyol, Hesperetin, Eriocitrin, Hesperidin, Naringin, Narirutin, Neoeriocitrin, Neohesperidin, Poncirin, Didymin, Narirutin 4′-O-glucoside, Naringin 4′-O-glucoside, Naringin 6′-malonate, Isosakuranetin, Naringenin 7-O-glucoside, Pinocembrin, 8-Prenylnaringenin, 6-Prenylnaringenin, 6-Geranylnaringenin, Isoxanthohumol, Eriodictyol 7-O-glucoside, Sakuranetin, Hesperetin 3′-O-glucuronide, Hesperetin 7-O-glucuronide, Hesperetin 3′-sulfate, Homoeriodictyol, Naringenin 4′-O-glucuronide, Naringenin 5-O-glucuronide, Naringenin 7-O-glucuronide, Hesperetin 3′,7-O-diglucuronide, and Hesperetin 5,7-O-diglucuronide.

In some embodiments, the different types of polyphenol comprise flavones. In some embodiments, the different types of polyphenol comprise Total flavones (i.e. the flavones are grouped together and classified as one “type” of polyphenol). In some embodiments, the different types of polyphenol comprise one or more flavone. “Flavones” may include flavonoids that have the backbone of 2-phenylchromen-4-one. Exemplary flavones include: Apigenin, Luteolin, Diosmin, Isorhoifolin, Neodiosmin, Rhoifolin, Sinensetin, Nobiletin, Tangeretin, Luteolin 7-O-diglucuronide, Chrysin, Luteolin 7-O-rutinoside, Tetramethylscutellarein, Luteolin 7-O-glucoside, Apigenin 7-O-glucoside, Apigenin 6,8-di-C-glucoside, Apigenin 6,8-C-arabinoside-C-glucoside, Apigenin 6,8-C-galactoside-C-arabinoside, Luteolin 7-O-glucuronide, Apigenin 7-O-glucuronide, Luteolin 7-O-malonyl-glucoside, Luteolin 6-C-glucoside, Luteolin 7-O-(2-apiosyl-glucoside), Luteolin 7-O-(2-apiosyl-6-malonyl)-glucoside, Apigenin 7-O-apiosyl-glucoside, 7,3′,4′-Trihydroxyflavone, 7,4′-Dihydroxyflavone, Geraldone, Baicalein, Apigenin 6-C-glucoside, Hispidulin, Cirsimaritin, 5,6-Dihydroxy-7,8,3′,4′-tetramethoxyflavone, Pebrellin, Gardenin B, Nepetin, Jaceosidin, Cirsilineol, Eupatorin, 6-Hydroxyluteolin, 6-Hydroxyluteolin 7-O-rhamnoside, Scutellarein, Apigenin 7-O-(6″-malonyl-apiosyl-glucoside), Chrysoeriol 7-O-apiosyl-glucoside, Chrysoeriol 7-O-(6″-malonyl-apiosyl-glucoside), Chrysoeriol 7-O-glucoside, Chrysoeriol 7-O-(6″-malonyl-glucoside), Apigenin 7-O-diglucuronide, and Rhoifolin 4′-O-glucoside.

In some embodiments, the different types of polyphenol comprise flavonols. In some embodiments, the different types of polyphenol comprise Total flavonols (i.e. the flavonols are grouped together and classified as one “type” of polyphenol). In some embodiments, the different types of polyphenol comprise one or more flavonol. “Flavonols” may include flavonoids that have the 3-hydroxyflavone backbone. Exemplary flavonols include:

Kaempferol, Quercetin, Quercetin 3-O-galactoside, Quercetin 3-O-glucoside, Quercetin 3-O-xyloside, Quercetin 3-O-rhamnoside, Quercetin 3-O-rutinoside, Quercetin 3-O-sophoroside, Quercetin 3-O-arabinoside, Quercetin 3-O-xylosyl-glucuronide, Isorhamnetin 3-O-glucoside 7-O-rhamnoside, Isorhamnetin 3-O-rutinoside, Kaempferol 3-O-glucuronide, Isorhamnetin 7-O-rhamnoside, Quercetin 3,4′-O-diglucoside, Myricetin 3-O-rutinoside, Myricetin, Morin, Kaempferide, Myricetin 3-O-galactoside, Myricetin 3-O-glucoside, Quercetin 3-O-glucosyl-xyloside, Quercetin 3-O-acetyl-rhamnoside, Kaempferol 3-O-galactoside, Galangin, Isorhamnetin, Kaempferol 3-O-glucoside, Kaempferol 3-O-rutinoside, Kaempferol 3-O-glucosyl-rhamnosyl-galactoside, Kaempferol 3-O-glucosyl-rhamnosyl-glucoside, Quercetin 3-O-glucosyl-rhamnosyl-galactoside, Quercetin 3-O-glucosyl-rhamnosyl-glucoside, Rhamnetin, Isorhamnetin 3-O-glucoside, Myricetin 3-O-rhamnoside, Quercetin 3-O-rhamnosyl-galactoside, Quercetin 3-O-glucuronide, Isorhamnetin 3-O-glucuronide, Myricetin 3-O-arabinoside, Quercetin 7,4′-O-diglucoside, Quercetin 4′-O-glucoside, Isorhamnetin 4′-O-glucoside, 3,7-Dimethylquercetin, Kaempferol 3-O-sophoroside, Kaempferol 3,7-O-diglucoside, Kaempferol 3-O-sophoroside 7-O-glucoside, Quercetin 3-O-(6″-malonyl-glucoside), Kaempferol 3-O-(6″-malonyl-glucoside), Kaempferol 3-O-rhamnoside, Quercetin 3-O-(6″-malonyl-glucoside) 7-O-glucoside, Patuletin 3-O-glucosyl-(1->6)-[apiosyl(1->2)]-glucoside, Spinacetin 3-O-glucosyl-(1->6)-[apiosyl(1->2)]-glucoside, Patuletin 3-O-(2″-feruloylglucosyl)(1->6)-[apiosyl(1->2)]-glucoside, Spinacetin 3-O-(2″-p-coumaroylglucosyl)(1->6)-[apiosyl(1->2)]-glucoside, Spinacetin 3-O-(2″-feruloylglucosyl)(1->6)-[apiosyl(1->2)]-glucoside, Spinacetin 3-O-glucosyl-(1->6)-glucoside, Jaceidin 4′-O-glucuronide, 5,3′,4′-Trihydroxy-3-methoxy-6:7-methylenedioxyflavone 4′-O-glucuronide, 5,4′-Dihydroxy-3,3′-dimethoxy-6:7-methylenedioxyflavone 4′-O-glucuronide, Kaempferol 3-O-xylosyl-glucoside, Kaempferol 3-O-acetyl-glucoside, Quercetin 3-O-xylosyl-rutinoside, Kaempferol 3-O-xylosyl-rutinoside, Kaempferol 7-O-glucoside, Kaempferol 3-O-galactoside 7-O-rhamnoside, Kaempferol 3-O-(6″-acetyl-galactoside) 7-O-rhamnoside, Quercetin 3-O-galactoside 7-O-rhamnoside, Quercetin 3-O-(6″-acetyl-galactoside) 7-O-rhamnoside, Kaempferol 3-O-(2″-rhamnosyl-galactoside) 7-O-rhamnoside, Kaempferol 3-O-(2″-rhamnosyl-6″-acetyl-galactoside) 7-O-rhamnoside, 6,8-Dihydroxykaempferol, Isorhamnetin 3-O-galactoside, Quercetin 3-O-rhamnosyl-rhamnosyl-glucoside, Kaempferol 3-O-rhamnosyl-rhamnosyl-glucoside, Methylgalangin, Kaempferol 3,7,4′-O-triglucoside, 3-Methoxynobiletin, 3-Methoxysinensetin, Quercetin 3′-O-glucuronide, Quercetin 3′-sulfate, Quercetin 4′-O-glucuronide, and Isorhamnetin 4′-O-glucuronide.

In some embodiments, the different types of polyphenol comprise isoflavonoids. In some embodiments, the different types of polyphenol comprise Total isoflavonoids (i.e. the isoflavonoids are grouped together and classified as one “type” of polyphenol). In some embodiments, the different types of polyphenol comprise one or more isoflavonoid. “Isoflavonoids” may have the 3-phenylchromen-4-one backbone with no hydroxyl group substitution at position 2 or the 3-phenylchroman (isoflavan) backbone. Exemplary isoflavonoids include Daidzein, Formononetin, Genistein, Biochanin A, Glycitein, Glycitin, 6″-O-Acetyldaidzin, 6″-O-Malonylgenistin, Daidzin, Genistin, 6″-O-Acetylgenistin, 6″-O-Acetylglycitin, 6″-O-Malonyldaidzin, 6″-O-Malonylglycitin, 2′, 7-Dihydroxy-4′,5′-dimethoxyisoflavone, 2-Dehydro-O-desmethylangolensin, 2′-Hydroxyformononetin, 3′,4′,7-Trihydroxyisoflavan, 3′,4′, 7-Trihydroxyisoflavanone, 3′-Hydroxydaidzein, 3′-Hydroxy-O-desmethylangolensin, 4′,6,7-Trihydroxyisoflavanone, 4′,7-Dihydroxy-3′-methoxyisoflavan, 4′,7-Dihydroxy-6-methoxyisoflavan, 4′-O-Methylequol, 5,6,7,3′,4′-Pentahydroxyisoflavone, 5,6,7,4′-Tetrahydroxyisoflavone, 5,7,8,3′,4′-Pentahydroxyisoflavone, 5,7,8,4′-Tetrahydroxyisoflavone, 5′-Hydroxy-O-desmethylangolensin, 5′-Methoxy-O-desmethylangolensin, 6,7,3′,4′-Tetrahydroxyisoflavone, 6,7,4′-Trihydroxyisoflavone, 6′-Hydroxyangolensin, 6′-Hydroxy-O-desmethylangolensin, 7,8,3′,4′-Tetrahydroxyisoflavone, 7,8,4′-Trihydroxyisoflavone, Angolensin, Calycosin, Daidzein 4′-O-glucuronide, Daidzein 7-O-glucuronide, Dihydrobiochanin A, Dihydrodaidzein, Dihydrodaidzein 7-O-glucuronide, Dihydroformononetin, Dihydrogenistein, Dihydroglycitein, Equol, Formononetin 7-O-glucuronide, Genistein 4′,7-O-diglucuronide, Genistein 4′-O-glucuronide, Genistein 5-O-glucuronide, Genistein 7-O-glucuronide, Glycitein 4′-O-glucuronide, Glycitein 7-O-glucuronide, Koparin, O-Desmethylangolensin, Orobol, Prunetin, Pseudobaptigenin, Puerarin, Daidzin 4′-O-glucuronide, Irisolidone 7-O-glucuronide, Tectorigenin 7-sulfate, Tectorigenin 4′-sulfate, Irisolidone, Tectorigenin, Tectoridin, 5,7-Dihydroxy-8,4′-dimethoxyisoflavone, Isotectorigenin, Equol 7-O-glucuronide, Equol 4′-O-glucuronide, 3′,4′,5,7-Tetrahydroxyisoflavanone, 3′-O-Methylequol, 6-O-Methylequol, 3′-Hydroxygenistein, 6-Hydroxydihydrodaidzein, 3′-Hydroxyequol, cis-4-Hydroxyequol, 4′-Methoxy-2′,3,7-trihydroxyisoflavanone, Irilone, Vestitone, Sativanone, Butin, 3′-Hydroxymelanettin, Melanettin, Stevenin, Violanone, Isoliquiritigenin, Dalbergin, 3′-O-Methylviolanone, and 8-Hydroxydihydrodaidzein. In some embodiments, the different types of polyphenol comprise one or more class of isoflavonoid. Exemplary classes of isoflavonoids include isoflavones, isoflavonones, isoflavanes, isoflavandiols, isoflavenes, and pterocarpans. In some embodiments, the different types of polyphenol comprise one or more of: Total isoflavones, Total isoflavonones, Total isoflavanes, Total isoflavandiols, Total isoflavenes, and Total pterocarpans (i.e. each of the isoflavones, isoflavonones, isoflavanes, isoflavandiols, isoflavenes, and/or pterocarpans are separately grouped together and each classified as one “type” of polyphenol).

### Lignans

In some embodiments, the different types of polyphenol comprise lignans and/or neolignans. In some embodiments, the different types of polyphenol comprise Total lignans and/or neolignans (i.e. the lignans and/or neolignans are grouped together and classified as one “type” of polyphenol). In some embodiments, the different types of polyphenol comprise one or more lignin and/or neolignan.

“Lignans” and “neolignans” are a large group of natural products characterised by the coupling of two C₆C₃ units. The term lignan was introduced for the structures where the two units are β,β′-linked. When the two are coupled in other ways (m,m′; γ,γ′; β,m′; etc.) they are called neolignans (Moss G.P., et al., 2000. Pure and Applied Chemistry, 72(8), pp. 1493-1523).

Exemplary lignans include Secoisolariciresinol, Matairesinol, Lariciresinol, Pinoresinol, Syringaresinol, Isolariciresinol, Arctigenin, Trachelogenin, Medioresinol, 1-Acetoxypinoresinol, Sesamin, Sesamolin, Sesamolinol, Sesaminol, Sesamol, 7-Hydroxymatairesinol, Isohydroxymatairesinol, Secoisolariciresinol-sesquilignan, Cyclolariciresinol, 7-Oxomatairesinol, Todolactol A, Conidendrin, 7-Hydroxysecoisolariciresinol, Nortrachelogenin, Lariciresinol-sesquilignan, Anhydro-secoisolariciresinol, Dimethylmatairesinol, Episesamin, Episesaminol, Enterodiol, Enterolactone, Sesaminol 2-O-triglucoside, Schisandrin, Gomisin D, Schisandrol B, Tigloylgomicin H, Schisanhenol, Schisantherin A, Gomisin M2, Deoxyschisandrin, Schisandrin B, Schisandrin C, 2-Hydroxyenterodiol, 4-Hydroxyenterodiol, 6-Hydroxyenterodiol, 2-Hydroxyenterolactone, 4-Hydroxyenterolactone, 6-Hydroxyenterolactone, 2′-Hydroxyenterolactone, 4′-Hydroxyenterolactone, 6′-Hydroxyenterolactone, 5-Hydroxyenterolactone, and 7-Hydroxyenterolactone.

### Stilbenes

In some embodiments, the different types of polyphenol comprise stilbenes. In some embodiments, the different types of polyphenol comprise Total stilbenes (i.e. the stilbenes are grouped together and classified as one “type” of polyphenol). In some embodiments, the different types of polyphenol comprise one or stilbene.

The term “stilbene” may refer to both stereoisomers of 1,2-diphenylethene and derivatives thereof (e.g. stilbenoids).

Exemplary stilbenes include: Trans-Resveratrol, Piceatannol, e-Viniferin, Pterostilbene, d-Viniferin, Pallidol, Piceatannol 3-O-glucoside, Pinosylvin, Resveratrol 5-O-glucoside, Resveratrol, Resveratrol 3-O-glucoside, 3,4,5,4′-Tetramethoxystilbene, 3′-Hydroxy-3,4,5,4′-tetramethoxystilbene, 4′-Hydroxy-3,4,5-trimethoxystilbene, 4-Hydroxy-3,5,4′-trimethoxystilbene, cis-Resveratrol 3-O-glucuronide, cis-Resveratrol 3-sulfate, cis-Resveratrol 4′-O-glucuronide, cis-Resveratrol 4′-sulfate, Resveratrol 3-sulfate, trans-Resveratrol 3,5-disulfate, trans-Resveratrol 3,4′-disulfate, trans-Resveratrol 3-O-glucuronide, trans-Resveratrol 3-sulfate, trans-Resveratrol 4′-O-glucuronide, trans-Resveratrol 4′-sulfate, and Dihydroresveratrol.

### Phenolic Acids

In some embodiments, the different types of polyphenol comprise phenolic acids. In some embodiments, the different types of polyphenol comprise Total phenolic acids (i.e. the phenolic acids are grouped together and classified as one “type” of polyphenol). “Phenolic acids” may include products containing a phenolic ring and an organic carboxylic acid function and may include hydroxybenzoic acids, hydroxycinnamic acids, hydroxyphenylacetic acids, hydroxyphenylpropanoic acids, and hydroxyphenylpentanoic acids.

In some embodiments, the different types of polyphenol comprise one or more class of phenolic acid. Exemplary classes of phenolic acids include hydroxybenzoic acids, hydroxycinnamic acids, hydroxyphenylacetic acids, hydroxyphenylpropanoic acids, and hydroxyphenylpentanoic acids. In some embodiments, the different types of polyphenol comprise one or more of: Total hydroxybenzoic acids, Total hydroxycinnamic acids, Total hydroxyphenylacetic acids, Total hydroxyphenylpropanoic acids, and Total hydroxyphenylpentanoic acids.

In some embodiments, the different types of polyphenol comprise hydroxybenzoic acids. In some embodiments, the different types of polyphenol comprise Total hydroxybenzoic acids (i.e. the hydroxybenzoic acids are grouped together and classified as one “type” of polyphenol). In some embodiments, the different types of polyphenol comprise one or more hydroxybenzoic acid. “Hydroxybenzoic acids” may include 2-Hydroxybenzoic acid, 3-Hydroxybenzoic acid, 4-Hydroxybenzoic acid, and derivatives thereof. Exemplary hydroxybenzoic acids include: Ellagic acid glucoside, Protocatechuic acid, Gallic acid, Vanillic acid, Gentisic acid, Ellagic acid, 4-Hydroxybenzoic acid, Syringic acid, 5-O-Galloylquinic acid, Ellagic acid arabinoside, Ellagic acid acetyl-xyloside, Ellagic acid acetyl-arabinoside, Benzoic acid, 2-Hydroxybenzoic acid, 3-Hydroxybenzoic acid, 2,3-Dihydroxybenzoic acid, 2,4-Dihydroxybenzoic acid, 4-Hydroxybenzoic acid 4-O-glucoside, Protocatechuic acid 4-O-glucoside, Gallic acid 4-O-glucoside, 3,5-Dihydroxybenzoic acid, 2,6-Dihydroxybenzoic acid, Gallic acid 3-O-gallate, Gallic acid ethyl ester, Valoneic acid dilactone, Galloyl glucose, Lambertianin C, Sanguiin H-6, Punicalagin, Gallagic acid, 3-O-Methylgallic acid, 4-O-Methylgallic acid, 3,4-O-Dimethylgallic acid, Punicalin, 4-Hydroxyhippuric acid, 3-Hydroxyhippuric acid, 2-Hydroxyhippuric acid, Hippuric acid, Paeoniflorin, and Vanillic acid 4-sulfate.

In some embodiments, the different types of polyphenol comprise hydroxycinnamic acids. In some embodiments, the different types of polyphenol comprise Total hydroxycinnamic acids (i.e. the hydroxycinnamic acids are grouped together and classified as one “type” of polyphenol). In some embodiments, the different types of polyphenol comprise one or more hydroxycinnamic acid. “Hydroxycinnamic acids” may include hydroxyl derivatives of cinnamic acid having a C6-C3 skeleton. Exemplary hydroxycinnamic acids include: p-Coumaric acid, 5-p-Coumaroylquinic acid, 4-p-Coumaroylquinic acid, Caffeic acid, Feruloyl glucose, Ferulic acid, Caffeoyl tartaric acid, Rosmarinic acid, o-Coumaric acid, m-Coumaric acid, Sinapic acid, p-Coumaroyl glucose, p-Coumaroylquinic acid, 3-Caffeoylquinic acid, Verbascoside, 4-Caffeoylquinic acid, p-Coumaroyl tartaric acid, 2,5-di-S-Glutathionyl caftaric acid, Feruloyl tartaric acid, Caffeic acid ethyl ester, Cinnamoyl glucose, 5-Caffeoylquinic acid, 3-p-Coumaroylquinic acid, 2-S-Glutathionyl caftaric acid, 5-Feruloylquinic acid, 4-Feruloylquinic acid, 3-Feruloylquinic acid, 5-Sinapoylquinic acid, 4-Sinapoylquinic acid, 3-Sinapoylquinic acid, 3,5-Dicaffeoylquinic acid, Isoferulic acid, Caffeoyl glucose, p-Coumaric acid 4-O-glucoside, Caffeic acid 4-O-glucoside, Ferulic acid 4-O-glucoside, p-Coumaroyl tartaric acid glucosidic ester, p-Coumaric acid ethyl ester, Hydroxycaffeic acid, Chicoric acid, 5-5′-Dehydrodiferulic acid, 5-8′-Dehydrodiferulic acid, 1,2-Disinapoylgentiobiose, 1-Sinapoyl-2-feruloylgentiobiose, 1,2-Diferuloylgentiobiose, 1,2,2′-Trisinapoylgentiobiose, 1,2′-Disinapoyl-2-feruloylgentiobiose, 1-Sinapoyl-2,2′-diferuloylgentiobiose, 1,2,2′-Triferuloylgentiobiose, 8-O-4′-Dehydrodiferulic acid, 5-8′-Benzofuran dehydrodiferulic acid, 3,4-Dicaffeoylquinic acid, 3,4-Diferuloylquinic acid, 3,5-Diferuloylquinic acid, 1,5-Dicaffeoylquinic acid, 4,5-Dicaffeoylquinic acid, Avenanthramide 2p, Avenanthramide 2c, Avenanthramide 2f, p-Coumaroyl malic acid, p-Coumaroyl glycolic acid, Cinnamic acid, Caffeoyl aspartic acid, p-Coumaroyl tyrosine, Sinapine, Avenanthramide K, 24-Methylcholestanol ferulate, 24-Methylcholesterol ferulate, 24-Methyllathosterol ferulate, Stigmastanol ferulate, Sitosterol ferulate, Schottenol ferulate, 24-Methylenecholestanol ferulate, 3-O-Methylrosmarinic acid, Feruloylglycine, Isoferulic acid 3-O-glucuronide, Isoferulic acid 3-sulfate, Ferulic acid 4-sulfate, Ferulic acid 4-O-glucuronide, Caffeic acid 4-sulfate, Caffeic acid 3-sulfate, Feruloyl C1-glucuronide, Isoferuloyl C1-glucuronide, Caffeic acid 3-O-glucuronide, Caffeic acid 4-O-glucuronide, Caffeoyl C1-glucuronide, 1,5-Diferuloylquinic acid, 1-Caffeoyl-5-feruloylquinic acid, and 1-Feruloyl-5-caffeoylquinic acid.

In some embodiments, the different types of polyphenol comprise hydroxyphenylacetic acids. In some embodiments, the different types of polyphenol comprise Total hydroxyphenylacetic acids (i.e. the hydroxyphenylacetic acids are grouped together and classified as one “type” of polyphenol). In some embodiments, the different types of polyphenol comprise one or more hydroxyphenylacetic acid. “Hydroxyphenylacetic acids” may include hydroxyl derivatives of phenylacetic acid. Exemplary hydroxyphenylacetic acids include: 3,4-Dihydroxyphenylacetic acid, 4-Hydroxyphenylacetic acid, Homovanillic acid, Homoveratric acid, Methoxyphenylacetic acid, 3-Hydroxyphenylacetic acid, 2-Hydroxyphenylacetic acid, Phenacetylglycine, Phenylacetic acid, 4-Hydroxymandelic acid, 2-Hydroxy-2-phenylacetic acid, and Homovanillic acid 4-sulfate.

In some embodiments, the different types of polyphenol comprise hydroxyphenylpropanoic acids. In some embodiments, the different types of polyphenol comprise Total hydroxyphenylpropanoic acids (i.e. the hydroxyphenylpropanoic acids are grouped together and classified as one “type” of polyphenol). In some embodiments, the different types of polyphenol comprise one or more hydroxyphenylpropanoic acid. “Hydroxyphenylpropanoic acids” may include hydroxyl derivatives of phenylpropanoic acid. Exemplary hydroxyphenylpropanoic acids include: Dihydro-p-coumaric acid, Dihydrocaffeic acid, 3-Hydroxy-3-(3-hydroxyphenyl)propionic acid, 3-(3,4-Dihydroxyphenyl)-2-methoxypropionic acid, 3-Hydroxyphenylpropionic acid, Dihydroferulic acid 4-sulfate, Dihydrocaffeic acid 3-O-glucuronide, Dihydrocaffeic acid 3-sulfate, Dihydroferulic acid, Dihydroferulic acid 4-O-glucuronide, Dihydrosinapic acid, Dihydroferuloylglycine, Danshensu, 3-Methoxy-4-hydroxyphenyllactic acid, 3,4-Dihydroxyphenyllactic acid methyl ester, Hydroxydanshensu, 3-Phenylpropionic acid, 3-Hydroxy-4-methoxyphenyllactic acid, and 4-Hydroxyphenyl-2-propionic acid.

In some embodiments, the different types of polyphenol comprise hydroxyphenylpentanoic acids. In some embodiments, the different types of polyphenol comprise Total hydroxyphenylpentanoic acids (i.e. the hydroxyphenylpentanoic acids are grouped together and classified as one “type” of polyphenol). In some embodiments, the different types of polyphenol comprise one or more hydroxyphenylpentanoic acid. “Hydroxyphenylpentanoic acids may include hydroxyl derivatives of phenylpentanoic acid. Exemplary hydroxyphenylpentanoic acids include: 5-(3′-Methoxy-4′-hydroxyphenyl)-γ-valerolactone, 4-Hydroxy-(3′,4′-dihydroxyphenyl)valeric acid, 5-(3′,4′-dihydroxyphenyl)-valeric acid, 5-(3′,4′,-dihydroxyphenyl)-γ-valerolactone, 5-(3′,4′, 5′-trihydroxyphenyl)-γ-valerolactone, 5-(3′,5′-dihydroxyphenyl)-γ-valerolactone, 3-Hydroxyphenylvaleric acid, and 5-(3′,5′-dihydroxyphenyl)-γ-valerolactone 3-O-glucuronide.

### Other Polyphenols

The different types of phenol may comprise any other class of polyphenol. Other classes include: Alkylmethoxyphenols, Alkylphenols, Curcuminoids, Furanocoumarins, Hydroxybenzaldehydes, Hydroxybenzoketones, Hydroxycinnamaldehydes, Hydroxycoumarins, Hydroxyphenylpropenes, Methoxyphenols, Naphtoquinones, Phenolic terpenes, and Tyrosols. In some embodiments, the different types of polyphenol comprise one or more of: Total Alkylmethoxyphenols, Total Alkylphenols, Total Curcuminoids, Total Furanocoumarins, Total Hydroxybenzaldehydes, Total Hydroxybenzoketones, Total Hydroxycinnamaldehydes, Total Hydroxycoumarins, Total Hydroxyphenylpropenes, Total Methoxyphenols, Total Naphtoquinones, Total Phenolic terpenes, and Total Tyrosols (i.e. each of the Alkylmethoxyphenols, Alkylphenols, Curcuminoids, Furanocoumarins,

Hydroxybenzaldehydes, Hydroxybenzoketones, Hydroxycinnamaldehydes, Hydroxycoumarins, Hydroxyphenylpropenes, Methoxyphenols, Naphtoquinones, Phenolic terpenes, and/or Tyrosols are separately grouped together and each classified as one “type” of polyphenol).

The different types of phenol may comprise any other polyphenol. Other polyphenols include: 4-Vinylguaiacol, 4-Ethylguaiacol, 4-Vinylsyringol, 5-Heneicosenylresorcinol, 5-Heneicosylresorcinol, 5-Heptadecylresorcinol, 5-Nonadecenylresorcinol, 5-Nonadecylresorcinol, 5-Pentacosenylresorcinol, 5-Pentacosylresorcinol, 5-Pentadecylresorcinol, 5-Tricosenylresorcinol, 5-Tricosylresorcinol, 3-Methylcatechol, 4-Methylcatechol, 4-Ethylcatechol, 4-Vinylphenol, 4-Ethylphenol, Curcumin,

Demethoxycurcumin, Bisdemethoxycurcumin, Bergapten, Psoralen, Xanthotoxin, Isopimpinellin, Syringaldehyde, Protocatechuic aldehyde, Vanillin, 4-Hydroxybenzaldehyde, Gallic aldehyde, p-Anisaldehyde, Vanillin 4-sulfate, 3-Methoxyacetophenone, 2,3-Dihydroxy-1-guaiacylpropanone, Paeonol, 2,4-Dihydroxyacetophenone 5-sulfate, 2-Hydroxy-4-methoxyacetophenone 5-sulfate, Resacetophenone, Norathyriol, Ferulaldehyde,

Sinapaldehyde, Coumarin, Mellein, Scopoletin, Esculetin, Esculin, Umbelliferone, 4-Hydroxycoumarin, Urolithin A 3,8-O-diglucuronide, Urolithin A, Urolithin B, Urolithin B 3-O-glucuronide, Urolithin C, 2-Methoxy-5-prop-1-enylphenol, Anethole, Eugenol, Acetyl eugenol, [6]-Gingerol, Estragole, Guaiacol, Juglone, 1,4-Naphtoquinone, Carnosic acid, Rosmanol, Carnosol, Epirosmanol, Rosmadial, Thymol, Carvacrol, Tyrosol, Hydroxytyrosol, 3,4-DHPEA-AC, p-HPEA-AC, Oleuropein, Demethyloleuropein, 3,4-DHPEA-EA, Ligstroside, 3,4-DHPEA-EDA, Hydroxytyrosol 4-O-glucoside, Oleoside dimethylester, Oleoside 11-methylester, p-HPEA-EDA, p-HPEA-EA, Oleuropein-aglycone, Ligstroside-aglycone, Tyrosol 4-sulfate, Coumestrol, Catechol, Pyrogallol, Phlorin, Phenol, Arbutin, 3,4-Dihydroxyphenylglycol, Lithospermic acid, Salvianolic acid B, Salvianolic acid C, Salvianolic acid D, Salvianolic acid G, and Isopropyl 3-(3,4-dihydroxyphenyl)-2-hydroxypropanoate.

In some embodiments, the different types of polyphenol comprise one or more of: (−)-Epicatechin, (−)-Epicatechin 3-gallate, (−)-Epigallocatechin, (−)-Epigallocatechin 3-gallate, (+)-Catechin, (+)-Gallocatechin, Apigenin, Cyanidin, Daidzein, Delphinidin, Eriodictyol, Genistein, Glycitein, Hesperetin, Isorhamnetin, Kaempferol, Luteolin, Malvidin, Myricetin, Naringenin, Pelargonidin, Peonidin, Petunidin, Quercetin, Theaflavin, Theaflavin-3-gallate, Theaflavin-3,3′-digallate, Theaflavin-3′-gallate, and Thearubigins.

In some embodiments, the different types of polyphenol comprise one or more of: (−)-Epicatechin, (−)-Epicatechin 3-gallate, (−)-Epigallocatechin, (−)-Epigallocatechin 3-gallate, (+)-Catechin, (+)-Gallocatechin, Apigenin, Cyanidin, Daidzein, Delphinidin, Eriodictyol, Genistein, Glycitein, Hesperetin, Isorhamnetin, Kaempferol, Luteolin, Malvidin, Myricetin, Naringenin, Pelargonidin, Peonidin, Petunidin, Quercetin, Theaflavin, Theaflavin-3-gallate, Theaflavin-3,3′-digallate, Theaflavin-3′-gallate, Thearubigins, Total anthocyanidins, Total catechins, Total flavan-3-ols, Total flavanones, Total flavones, Total flavonoids, Total flavonols, and Total isoflavones.

### Polyphenol Content

Many common foods have had their polyphenol content determined. Phenol-Explorer is an online comprehensive database on polyphenol contents in foods, which can be used to analyse the polyphenol content of foods (see Pérez-Jiménez, J., et al., 2010. Journal of agricultural and food chemistry, 58(8), pp.4959-4969; and Pérez-Jiménez, J., et al., 2010. European journal of clinical nutrition, 64(3), pp.S112-S120). Phenol-Explorer provides the number of polyphenols for over 400 different foods including alcoholic beverages, cereals and cereal products, coffee and cocoa, fruits and fruit products, non-alcoholic beverages, oils, seasonings, seeds, and vegetables.

Routine methods for determining polyphenol content of food are known in the art, for example as described in Naczk, M. and Shahidi, F., 2004. Journal of chromatography A, 1054(1-2), pp.95-111; Ignat, I., et al., 2011. Food chemistry, 126(4), pp. 1821-1835; Kalili, K.M. and de Villiers, A., 2011. Journal of separation science, 34(8), pp.854-876; and López-Fernández, O., et al., 2020. Antioxidants, 9(6), p.479. Examples of suitable analytical techniques include liquid chromatography coupled to tandem mass spectrometry (LC-MS/MS) and liquid chromatography with diode array detector (LC-DAD).

### Polyphenol Diversity Index

As an alternative, or in addition, to determining the sum of different types of polyphenol present in the food product, meal, or diet, the methods of the present invention may comprise determining the diversity of polyphenols present in a food product, meal, or diet.

In one aspect, the present invention provides a method for determining the effects of a food product, meal, or diet on a subject's gut microbiota, wherein the method comprises determining the diversity of polyphenols present in a food product, meal, or diet. The method may further comprise determining the sum of different types of polyphenol present in the food product, meal, or diet.

The diversity of polyphenols present in a food product, meal, or diet may be determined by any suitable method.

In some embodiments, the diversity of polyphenols present in the food product, meal, or diet is determined using the sum of different types of polyphenol present in the food product, meal, or diet.

In some embodiments, the diversity of polyphenols present in the food product, meal, or diet is determined using the amount of each different type of polyphenol present in the food product, meal, or diet.

In some embodiments, the diversity of polyphenols present in the food product, meal, or diet is determined using the similarity in the chemical structure of the different types of polyphenol present in the food product, meal, or diet.

In some embodiments, the diversity of polyphenols present in the food product, meal, or diet is determined using the sum of different types of polyphenol present in the food product, meal, or diet; and the amount of each different type of polyphenol present in the food product, meal, or diet.

In some embodiments, the diversity of polyphenols present in the food product, meal, or diet is determined using the sum of different types of polyphenol present in the food product, meal, or diet; and the similarity in the chemical structure of the different types of polyphenol present in the food product, meal, or diet.

In some embodiments, the diversity of polyphenols present in the food product, meal, or diet is determined using the sum of different types of polyphenol present in the food product, meal, or diet; the amount of each different type of polyphenol present in the food product, meal, or diet; and the similarity in the chemical structure of the different types of polyphenol present in the food product, meal, or diet.

In some embodiments, the method of the present invention comprises determining the polyphenol richness of the food product, meal, or diet. “Polyphenol richness” may refer to the sum of different types of polyphenol present in the food product, meal, or diet.

In some embodiments, the method of the present invention comprises determining the polyphenol evenness of the food product, meal, or diet. “Polyphenol evenness” may refer to how close in content each polyphenol in the food product, meal, or diet is. The polyphenol evenness may be determined by any suitable statistical method known to the skilled person (e.g. using Pielou's evenness index). The amount of each different type of polyphenol present in the food product, meal, or diet may be used to determine the polyphenol evenness.

In some embodiments, the method of the present invention comprises determining a polyphenol diversity index for the food product, meal, or diet. A “polyphenol diversity index” may refer to a quantitative measure that reflects how many different types of polyphenol there are in the food product, meal, or diet, and that can simultaneously take into account the relations between those types, such as richness, evenness, or similarity in the chemical structure. The polyphenol diversity index may be determined by any suitable statistical method known to the skilled person (see e.g. Morris, E.K., et al., 2014. Ecology and evolution, 4(18), pp.3514-3524). For example, the polyphenol diversity index could be calculated using a non-phylogenetic measure (e.g. Shannon index) that accounts for polyphenol evenness; the polyphenol diversity index could be calculated using a phylogenetic measure (e.g Faith's PD diversity) that accounts for the similarity in chemical structure, where polyphenols would be mapped on a tree according to their chemical structure and mostly relying on the class (e.g. flavonoids) and sub-class (e.g. flavanols) that they belong to; or the polyphenol diversity index could be calculated using an abundance-weighted phylogenetic measure (see e.g. McCoy, C.O. and Matsen IV, F.A., 2013. PeerJ, 1, p.e157). The amount of each different type of polyphenol present in the food product, meal, or diet and/or the similarity in the chemical structure of the different types of polyphenol present in the food product, meal, or diet may be used to determine a polyphenol diversity index. In some embodiments, the polyphenol diversity index is a Shannon Index or a phylogenetic index (e.g. Faith's PD diversity).

### Microbiota and Microbiome

The inventors have shown that the diversity of polyphenol intake in a diet is associated with gut microbiome diversity.

A greater sum of different types of polyphenol present in the food product, meal, or diet (a greater polyphenol richness) may provide a more diverse gut microbiota. Additionally, a greater polyphenol evenness and/or a greater diversity of polyphenols (e.g. a greater polyphenol diversity index) may also provide a more diverse gut microbiota.

The “gut microbiota” is the composition of microorganisms (including bacteria, archaea and fungi) that live in the digestive tract. The term “gut microbiome” may encompass both the “gut microbiota” and their “theatre of activity”, which may include their structural elements (nucleic acids, proteins, lipids, polysaccharides), metabolites (signalling molecules, toxins, organic, and inorganic molecules), and molecules produced by coexisting hosts and structured by the surrounding environmental conditions (see e.g. Berg, G., et al., 2020. Microbiome, 8(1), pp.1-22). In the present invention, the term “gut microbiome” may therefore be used interchangeably with the term “gut microbiota”.

### Gut Microbiota Diversity

A subject's “gut microbiota diversity” may refer to the number of different taxa present in the gut microbiome and/or stool of the subject (e.g. “richness”). It may also refer to the “evenness” of the gut microbiome and/or stool of the subject, i.e. takes into account the abundance or relative abundance of each taxon.

Alpha diversity may be the diversity of a single sample (such as a fecal sample), and can take into account the number of different taxa and their relative abundances. Alpha diversity can be determined using a richness index, a phylogenetic diversity index, or a Shannon index. These indexes can be determined using methods routine in the art, such as, for example, using the R package Phyloseq (McMurdie and Holmes, 2013, PLOS One, 8, Article e61217). Alpha diversity indexes may be calculated based on 16 rRNA sequencing data and/or whole genome shotgun metagenomics sequencing.

Beta diversity can be determined using a Whittaker index (e.g. Jaccard or Sorensen), a Min-Max Index (e.g. Simpson, β-2 or β-3), a Cody Index or an Abundance index (e.g. Bray-Curtis or BD_{TOTAL}). These indexes can be determined using methods routine in the art, such as, for example, using the R package Phyloseq (McMurdie and Holmes, 2013, PLOS One, 8, Article e61217). Beta diversity indexes may be calculated based on 16 rRNA sequencing data and/or whole genome shotgun metagenomics sequencing.

In some embodiments of the invention, a more diverse gut microbiota has a higher alpha diversity, preferably wherein the alpha diversity is determined using a richness index, a phylogenetic diversity index (e.g. Faith's PD index), or a Shannon index.

In some embodiments of the invention, a higher dietary index provides a more diverse gut microbiota, preferably wherein a more diverse gut microbiota has a higher alpha diversity, more preferably wherein the alpha diversity is determined using a richness index, a phylogenetic diversity index (e.g. Faith's PD index), or a Shannon index.

### Gut Microbiota Data

The gut microbiota data of the subject may be obtained or obtainable by any suitable sampling method. For example, gut microbiota data may be obtained or obtainable by any method described in Tang, Q., et al., 2020, Frontiers in cellular and infection microbiology, 10, p.151.

The gut microbiota data may be obtained or obtainable from fecal samples, endoscopy samples (e.g. biopsy samples, luminal brush samples, laser capture microdissection samples), aspirated intestinal fluid samples, surgery samples, or by in vivo models or intelligent capsule (see e.g. Tang, Q., et al., 2020, Frontiers in cellular and infection microbiology, 10, p.151).

Suitably, the gut microbiota data may be obtained or obtainable from fecal samples. Fecal samples are naturally collected, non-invasive and can be sampled repeatedly. Fecal materials instantly frozen at −80° C. that can maintain microbial integrity without preservatives have been widely regarded as the gold standard for gut microbiota profiling, but other storage methods with or without preservatives can also be utilised to achieve microbiota compositions similar to those of fresh samples.

The gut microbiota data may be obtained or obtainable from the samples by any suitable detection method. For example, the gut microbiota data may be obtained by or obtainable by sequencing methods (e.g. next-generation sequencing (NGS) methods), PCR-based methods, semi-quantitative detection methods (e.g. from SwissDeCode), cycling temperature capillary electrophoresis (e.g. from REM analytics), cell-based methods, immunological-based methods, or any combination thereof. Preferably, the gut microbiota data is obtained by or obtainable by PCR-based methods, semi-quantitative detection methods (e.g. from SwissDeCode), cycling temperature capillary electrophoresis (e.g. from REM analytics), or immunological-based methods, or any combination thereof.

In some embodiments, the gut microbiota data is obtained or obtainable by sequencing methods (e.g. next-generation sequencing (NGS) methods). NGS enables the profiling of the genomic DNA of all the microorganisms present in a sample. NGS methods can include targeted (e.g. 16S ribosomal RNA sequencing) and/or shotgun sequencing approaches, e.g. as described in Poussin, C., et al., 2018. Drug discovery today, 23(9), pp.1644-1657.

In some embodiments, the gut microbiota data is obtained or obtainable by PCR-based methods. For example, the gut microbiota data may be obtained by or obtainable by PCR, multiplex PCR (mPCR), and/or quantitative PCR (qPCR). Suitably, the gut microbiota data may be obtained by or obtainable by qPCR, e.g. as described in Jian, C., et al., 2020. PLOS One, 15(1), p.e0227285.

In some embodiments, the gut microbiota data is obtained or obtainable by semi-quantitative detection methods. For example, the gut microbiota data may be obtained or obtainable by culture method, denaturing gradient gel electrophoresis (DGGE), terminal restriction fragment length polymorphism (T-RFLP), fluorescence in situ hybridization (FISH), and/or DNA microarrays, e.g. as described in Fraher, M.H., et al., 2012. Nature reviews Gastroenterology & hepatology, 9(6), p.312.

In some embodiments, the gut microbiota data is obtained or obtainable by cycling temperature capillary electrophoresis, e.g. as described in Refinetti, P., et al., 2016. Mitochondrion, 29, pp.65-74.

In some embodiments, the gut microbiota data may be obtained or obtainable by immunological-based methods. Immunological-based methods may be based on antibody-antigen interactions, whereby a particular antibody will bind to its specific antigen and can use polyclonal or monoclonal antibodies. Enzyme-linked immunosorbent assay (ELISA) and lateral flow immunoassay are among the immunological-based methods which can be used, e.g. as described in Law, J.W.F., et al., 2015. Frontiers in microbiology, 5, p.770. Exemplary methods are described in Amrouche, T., et al., 2006. Journal of microbiological methods, 65(1), pp. 159-170; and Qian, H., et al., 2008. Applied and environmental microbiology, 74(3), pp.833-839.

In some embodiments, the gut microbiota data is obtained or obtainable by cell-based methods. For example, the gut microbiota data may be obtained or obtainable by counting microbial cells using flow cytometry, e.g. as described in Galazzo, G., et al., 2020. Frontiers in cellular and infection microbiology, 10, p.403.

In some embodiments, the gut microbiota data is obtained or obtainable by a combination of one or more methods described herein, e.g. as described in Allaband, C., et al., 2019. Clinical Gastroenterology and Hepatology, 17(2), pp.218-230.

The gut microbiota data may provide the relative abundance and/or absolute abundance for the plurality of microbial taxa. Suitably, the gut microbiota data provides the relative abundance for the plurality of microbial taxa.

The microbial taxa may be classified according to a suitable classification, see e.g. Pitt, T.L. and Barer, M.R., 2012. Medical Microbiology, p.24. The microbial taxa may be taxonomically-classified and/or functionally-classified.

In some embodiments, the microbial taxa are taxonomically-classified. Microbial taxonomy refers to the rank-based classification of microbes. In the scientific classification established by Carl Linnaeus, each species has to be assigned to a genus, which in turn is a lower level of a hierarchy of ranks (family, suborder, order, subclass, class, division/phyla, kingdom and domain). Prokaryotic taxa which have been correctly described are reviewed in e.g. Bergey's manual of Systematic Bacteriology.

Suitably, the microbial taxa in the microbial ratios are taxonomically-classified by phylum, class, order, family, genus and/or species. Suitably, the microbial taxa in the microbial ratios are taxonomically-classified by phylum, genus and/or species. Suitably, the microbial taxa in the microbial ratios are taxonomically-classified by genus and/or species. In some embodiments, the microbial taxa in the microbial ratios are taxonomically-classified by genus. In some embodiments, the microbial taxa in the microbial ratios are taxonomically-classified by species.

In some embodiments, the microbial taxa are functionally-classified. For example, the microbial taxa may be classified by one or more phenotypic classification systems (e.g. gram stain, morphology, growth requirements, biochemical reactions, serologic systems, environmental reservoirs etc.). In some embodiments, the microbial taxa are classified according to biological or metabolic pathways, protein domains or families, functional modules, complex carbohydrate metabolism, antibiotic resistance, virulence factors, bacterial drug targets and endotoxins, mobile genetic elements, and/or any other functional properties, such as those described in Kultima, J.R., et al., 2016. Bioinformatics, 32(16), pp.2520-2523 and Overbeek, R., et al., 2014. Nucleic acids research, 42(D1), pp.D206-D214.

Suitably, the microbial taxa are bacterial taxa.

### Food Product, Meal or Diet

A “food product” may include a single food item consumed by the subject. A “meal” may include all the food products consumed in a single meal by the subject. The subject's “diet” may include all the food products consumed by the subject.

The subject's diet may provide a plurality of food groups. The term “food group” refers to a collection of foods that share similar nutritional properties or biological classifications. Nutrition guides typically divide foods into food groups and recommend daily servings of each group for a healthy diet. Exemplary food groups include fruits; vegetables; pulses, nuts or seeds; meats; starches or grains; dairy; and oils and fats.

The subject's diet may also provide a plurality of food types. The term “food type” may refer to a collection of foods from the same food group that share more similar nutritional properties or biological classifications. Each food group may be further grouped into a plurality of food types. Exemplary food types for the food group fruit can include apples, banana, citrus, berries, other fruits (e.g. pear, peach, pineapple), and dried fruits. Suitable food groups and food types can be readily determined by any suitable method known in the art. For example, suitable food groups and food types can be based on published observations (e.g. Dwyer JT. The Journal of Nutrition. 2018;148(suppl 3):1575S-80S or Phenol-Explorer).

### Subject

The subject may be any age. For example, the subject may be a child or an adult. The term “child” may refer to a subject aged under 18 years. The term “adult” may refer to a subject aged 18 years or older. In some embodiments, the subject is a child. In some embodiments, the subject is an adult.

In some embodiments, the subject is an infant, a toddler, or a young child. The term “infant” may refer to a subject aged from about 0 years to about 1 year. The term “toddler” may refer to a subject aged from about 1 year to about 3 years. The term “young child” may refer to a subject aged from about 3 years to about 5 years. In some embodiments, the infant, toddler, or young child is a preterm infant, toddler, or young child. A “preterm” or “premature”, toddler, or young child means an infant, toddler, or young child who was not born at term (e.g. born prior 36 weeks of gestation).

### Dietary Index

The methods of the present invention may comprise determining a dietary index. The dietary index may be any dietary index described herein.

In one aspect, the present invention provides a dietary index for assessing the effects of a food product, meal, or diet on a subject's gut microbiota. The dietary index may be a polyphenol dietary index. Such a dietary index may be determined by any method described herein.

A “dietary index” provides a summary measure of a characteristic for a food product, meal, or diet. A “polyphenol dietary index” provides a summary measure of polyphenols within a food product, meal or diet. A polyphenol dietary index according to the present invention is based, at least in part, on the sum of different types of polyphenol present in the food product, meal or diet.

Thus, the present invention may further comprise determining a dietary index using the sum of different types of polyphenol present in the food product, meal, or diet.

In some embodiments, the dietary index comprises or consists of the sum of different types of polyphenol present in the food product, meal, or diet.

In some embodiments, the amount of each different type of polyphenol present in the food product, meal, or diet and/or the similarity in the chemical structure of the different types of polyphenol present in the food product, meal, or diet.

In some embodiments, the dietary index is determined using the diversity of polyphenols present in the food product, meal, or diet.

In some embodiments, the dietary index is determined using polyphenol richness, polyphenol evenness, and/or a polyphenol diversity index.

In some embodiments, the dietary index comprises or consists of a polyphenol diversity index.

Suitably, a higher dietary index may provide a more diverse gut microbiota

In one aspect, the present invention provides a method for providing a dietary index. The dietary index may be determined by any method described herein. The dietary index may be any dietary index described herein.

### Methods for Adjusting a Food Product, Meal, or Diet

In one aspect, the present invention provides a method for adjusting a food product, meal, or diet.

The method for adjusting a food product, meal, or diet may comprise determining the effects of the food product, meal, or diet on a subject's gut microbiota using a method according to the present invention. For example, the method may comprise determining the polyphenol richness, polyphenol evenness, and/or a polyphenol diversity index for the food product, meal, or diet. In some embodiments, the method comprises determining a dietary index according to the present invention for the food product, meal, or diet.

The method may comprise adjusting the food product, meal, or diet to provide an adjusted food product, adjusted meal, or adjusted diet with an improved effect on the subject's gut microbiota diversity. The food product, meal, or diet may be adjusted to provide a greater diversity of polyphenols by any method known in the art. For example, the food product, meal, or diet may be enriched with phenolic compounds using any method known to the skilled person (see e.g. McDougall, G.J., 2017. Proceedings of the Nutrition Society, 76(2), pp.163-171). Methods for providing polyphenol-fortified foods are known in the art (see e.g. Sridhar, K. and Charles, A.L., 2021. International Journal of Food Science & Technology, 56(8), pp.3742-3751; and Roopchand, D.E., et al., 2012. Food Chemistry, 131(4), pp.1193-1200).

In some embodiments, the adjusted food product, adjusted meal, or adjusted diet provides a greater number of different types of polyphenol than the un-adjusted food product, un-adjusted meal, or un-adjusted diet.

In some embodiments, the adjusted food product, adjusted meal, or adjusted diet has a greater polyphenol richness, greater polyphenol evenness, and/or a polyphenol diversity index than the un-adjusted food product, un-adjusted meal, or un-adjusted diet.

In some embodiments, the adjusted food product, adjusted meal, or adjusted diet has a greater polyphenol diversity index than the un-adjusted food product, un-adjusted meal, or un-adjusted diet.

In some embodiments, the adjusted food product, adjusted meal, or adjusted diet has an improved dietary index than the un-adjusted food product, un-adjusted meal, or un-adjusted diet.

### Methods for Maintaining or Improving Gut Microbiota Diversity

In one aspect, the present invention provides a method for maintaining or improving gut microbiota diversity.

The method for maintaining or improving gut microbiota diversity may comprise determining the effects of the subject's present diet on the subject's gut microbiota using a method according to the present invention. For example, the method may comprise determining the polyphenol richness, polyphenol evenness, and/or a polyphenol diversity index for the subject's present diet. In some embodiments, the method comprises determining a dietary index according to the present invention for the subject's present diet.

The method may comprise adjusting the subject's present diet to provide an adjusted diet with an improved effect on the subject's gut microbiota diversity. The diet may be adjusted to provide a greater diversity of polyphenols by any method known in the art.

In one aspect, the present invention provides a method for maintaining or improving gut microbiota diversity, wherein the method comprises:
(a) determining the effects of the subject's present diet on the subject's gut microbiota using a method according to the present invention; and
(b) adjusting the subject's present diet to provide an adjusted diet with an improved effect on the subject's gut microbiota diversity.

In one aspect, the present invention provides a method for maintaining or improving gut microbiota diversity, wherein the method comprises:
(a) determining a dietary index according to the present invention for the subject's present diet; and
(b) adjusting the subject's present diet to provide an adjusted diet with an improved dietary index.

In some embodiments, the adjusted diet provides a greater number of different types of polyphenol than the subject's present diet.

In some embodiments, the adjusted diet has a greater polyphenol richness, greater polyphenol evenness, and/or a polyphenol diversity index than the subject's present diet.

In some embodiments, the adjusted diet has a greater polyphenol diversity index than the subject's present diet.

In some embodiments, the adjusted diet has an improved dietary index compared to the subject's present diet.

### Maintaining or Improving Gut Microbiota Diversity

Maintaining gut microbiota diversity may refer to the gut microbiota diversity not being significantly reduced. Improving gut microbiota diversity may refer to increasing the gut microbiota diversity.

An improved gut microbiota diversity may refer to a more “rich” and/or “even” gut microbiota, for example as determined by an Alpha diversity index such as a richness index, a phylogenetic diversity index (e.g. Faith's PD), or a Shannon index.

Suitably, after adjusting the diet of the subject, the subject may have a more diverse gut microbiota.

Improving the gut microbiota diversity may improve the functioning of the gut microbiome.

In some embodiments, after adjusting the diet, the subject may be in an appropriate gut maturation state, in an appropriate gut progression state, and/or in an appropriate gut succession stage. An “appropriate gut maturation state” may mean that the subject's gut microbiota is maturing normally or properly. An “appropriate gut progression state” may mean that the subject's gut microbiota is progressing or evolving in a timely manner. An “appropriate gut succession state” may mean that the subject's gut microbiota is succeeding in a timely manner.

In some embodiments, after adjusting the diet, the subject may be at decreased risk of suffering a disease, disorder or condition associated with the gut microbiome, such as such as Irritable Bowel Syndrome, Inflammatory Bowel Disease, allergy, diabetes, cancer, asthma, and obesity. In some embodiments, after adjusting the diet, the subject may be at decreased risk of suffering a disease, disorder or condition associated with fibre intake, such as cardiovascular disease, constipation, diverticular disease, oesophageal cancer, gastric cancer, colorectal adenomas and colorectal cancer, breast cancer, endometrial cancer, prostate cancer, pancreatic cancer, ovarian cancer, or renal cancer.

In some embodiments, after adjusting the diet, the subject may experience reduced symptoms of a disease, disorder or condition associated with the gut microbiome, such as such as Irritable Bowel Syndrome, Inflammatory Bowel Disease, allergy, diabetes, cancer, asthma, and obesity. In some embodiments, after adjusting the diet, the subject may experience reduced symptoms of a disease, disorder or condition associated with fibre intake, such as cardiovascular disease, constipation, diverticular disease, oesophageal cancer, gastric cancer, colorectal adenomas and colorectal cancer, breast cancer, endometrial cancer, prostate cancer, pancreatic cancer, ovarian cancer, or renal cancer.

### Use

The inventors have shown that the diversity of polyphenols in a food product, meal, or diet may be used to assess the effects of the food product, meal, or diet on a subject's gut microbiota.

In one aspect, the present invention provides use of the sum of different types of polyphenol present in a food product, meal, or diet to assess the effects of the food product, meal, or diet on a subject's gut microbiota.

In another aspect, the present invention provides use of polyphenol richness, polyphenol evenness, and/or a polyphenol diversity index to assess the effects of the food product, meal, or diet on a subject's gut microbiota.

In another aspect, the present invention provides use of a dietary index according to the present invention for assessing the effects of a food product, meal, or diet on a subject's gut microbiota.

The uses of the present invention may comprise any steps included in the methods described herein.

### Computer Programs and Computer-Readable Mediums

The methods described herein may be computer-implemented methods.

In one aspect, the present invention provides a data processing system comprising means for carrying out a method of the invention.

In one aspect, the present invention provides a data processing apparatus comprising a processor configured to perform a method of the invention.

In one aspect, the present invention provides a computer program comprising instructions which, when the program is executed by a computer, cause the computer to carry out a method of the invention.

In one aspect, the present invention provides a computer-readable medium comprising instructions which, when executed by a computer, cause the computer to carry out a method of the invention.

In one aspect, the present invention provides a computer-readable data carrier having stored thereon the computer program of the invention.

In one aspect, the present invention provides a data carrier signal carrying the computer program of the invention.

The systems described herein may display a dashboard or other appropriate user interface to a user that is customized based on the food product, meal, or diet of interest. For example, based on the diversity of polyphenols in the food product, meal, or diet, the system may provide recommendations to adjust the food product, meal, or diet.

### Determining a Dietary Index

In one aspect, the present invention provides a data processing system comprising means for determining a dietary index for assessing the effects of a food product, meal, or diet on a subject's gut microbiota, given the different types of polyphenol present in the food product, meal, or diet.

In one aspect, the present invention provides a data processing apparatus comprising a processor configured to determine a dietary index for assessing the effects of a food product, meal, or diet on a subject's gut microbiota, given the different types of polyphenol present in the food product, meal, or diet.

In one aspect, the present invention provides a computer program comprising instructions which, when the program is executed by a computer, cause the computer to determine a dietary index for assessing the effects of a food product, meal, or diet on a subject's gut microbiota, given the different types of polyphenol present in the food product, meal, or diet.

In another aspect, the present invention provides a computer-readable medium comprising instructions which, when executed by a computer, cause the computer to determine a dietary index for assessing the effects of a food product, meal, or diet on a subject's gut microbiota, given the different types of polyphenol present in the food product, meal, or diet.

### Adjusting a Food Product, Meal, or Diet

In one aspect, the present invention provides a data processing system comprising means for adjusting a food product, meal, or diet, given the different types of polyphenol present in the food product, meal, or diet.

In one aspect, the present invention provides a data processing apparatus comprising a processor configured to adjust a food product, meal, or diet, given the different types of polyphenol present in the food product, meal, or diet.

In one aspect, the present invention provides a computer program comprising instructions which, when the program is executed by a computer, cause the computer to adjust a food product, meal, or diet, given the different types of polyphenol present in the food product, meal, or diet.

In another aspect, the present invention provides a computer-readable medium comprising instructions which, when executed by a computer, cause the computer to adjust a food product, meal, or diet, given the different types of polyphenol present in the food product, meal, or diet.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E.F. and Maniatis, T. (1989) Molecular Cloning: A Laboratory Manual, 2nd Edition, Cold Spring Harbor Laboratory Press; Ausubel, F.M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J.M. and McGee, J.O′D. (1990) In Situ Hybridization: Principles and Practice, Oxford University Press; Gait, M.J. (1984) Oligonucleotide Synthesis: A Practical Approach, IRL Press; and Lilley, D.M. and Dahlberg, J.E. (1992) Methods in Enzymology: DNA Structures Part A: Synthesis and Physical Analysis of DNA, Academic Press.

### EXAMPLES

Preferred features and embodiments of the invention will now be described by way of non-limiting examples.

### Example 1—Diversity of Dietary Polyphenol Intake is Associated With Gut Microbiome Diversity

### Material and Methods

We used publicly available American gut project (AGP) microbiome and dietary data collected on 1777 participants. A polyphenol diversity index was constructed based on the presence or absence of 38 polyphenols in the 259 food items present in the food frequency questionnaire (FFQ) distributed to AGP participants (table 1). A polyphenol was considered as part of a participant's diet if present (according to USDA) in at least one of the food items consumed. The polyphenol diversity index was calculated as the sum of all polyphenols consumed by a participant.

We next explored the association between the polyphenol diversity index and gut microbiome diversity (observed taxa, Shannon and faith PD) using linear mixed effect models adjusting for age, gender, BMI and season and where gut microbiome indexes were positioned as response and polyphenols as predictors. To assess the effects of fibre on the results, the same analysis was repeated including total fibre intake as a covariate.

TABLE 1
List of polyphenols identified in the AGP FFQs.

| | Number of |
|---|---|
| Polyphenol | foods from FFQs |
| (−)-Epicatechin | 18 |
| (−)-Epicatechin 3-gallate | 4 |
| (−)-Epigallocatechin | 9 |
| (−)-Epigallocatechin 3-gallate | 6 |
| (+)-Catechin | 17 |
| (+)-Gallocatechin | 6 |
| Apigenin | 12 |
| Cyanidin | 9 |
| Daidzein | 6 |
| Delphinidin | 4 |
| Eriodictyol | 4 |
| Genistein | 9 |
| Glycitein | 3 |
| Hesperetin | 6 |
| Isorhamnetin | 13 |
| Kaempferol | 39 |
| Luteolin | 33 |
| Malvidin | 3 |
| Myricetin | 28 |
| Naringenin | 8 |
| Pelargonidin | 2 |
| Peonidin | 5 |
| Petunidin | 2 |
| Quercetin | 46 |
| Theaflavin | 2 |
| Theaflavin-3-gallate | 2 |
| Theaflavin-3,3′-digallate | 2 |
| Theaflavin-3′-gallate | 2 |
| Thearubigins | 2 |
| Total anthocyanidins | 10 |
| Total catechins (monomeric flavan-3-ols only) | 21 |
| Total flavan-3-ols | 21 |
| Total flavanones | 8 |
| Total flavones | 34 |
| Total flavonoids | 58 |
| Total flavonols | 47 |
| Total isoflavones | 9 |

### Results

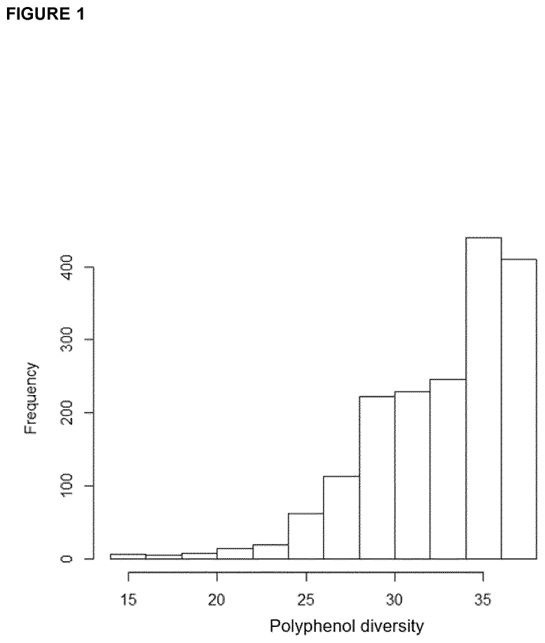

The polyphenol diversity index ranged between 15 and 37 in the AGP population with a median of 34 and a mean of 33 (FIG. 1).

We observed a significant positive association between gut microbiome diversity and dietary polyphenol diversity. The most significant result was obtained with the number of observed taxa (beta=0.85; SE=0.22 and P=1.0×10⁻⁴) followed by Faith PD (beta=0.08; SE=0.02 and P=2×10⁻⁴) and Shannon (beta=0.016; SE=0.005 and P=2×10⁻³). Additionally, we observed a positive association between polyphenol diversity and the level of butyrate producing bacteria (beta=0.0015; SE=0.0006 and P=0.019). Since most polyphenol containing foods are also rich in fibre that is known to affect the composition of the gut microbiome, we next questioned the role of fibre on the above results by adjusting the above models for total fibre intake. All associations between polyphenol and microbiome diversity remained significant (Shannon: beta=0.014, SE=0.005, P=9.4×10^{-3;} Richness: beta=0.76, SE=0.22, P=7.4×10^{-4;} Faith: beta=0.075, SE=0.022, P=5.6×10⁻⁴). However, the association between butyrate producing bacteria and polyphenol diversity was not significant (P=0.13).

The newly polyphenol diversity index can be used to capture the diversity of polyphenol intake in a diet and is associated with gut microbiome diversity. This could help to capture the level of polyphenol diversity present in a food product, meal, or diet, to help consumers to improve their polyphenol intake and potentially boost their gut microbiome diversity.

### EMBODIMENTS

Various preferred features and embodiments of the present invention will now be described with reference to the following numbered paragraphs (paras).
1. A method for determining the effects of a food product, meal, or diet on a subject's gut microbiota, wherein the method comprises determining the sum of different types of polyphenol present in the food product, meal, or diet.
2. The method according to para 1, wherein the method further comprises determining the amount of each different type of polyphenol present in the food product, meal, or diet.
3. The method according to para 1 or 2, wherein the method comprises determining the diversity of polyphenols present in the food product, meal, or diet, preferably wherein the diversity of polyphenols present in the food product, meal, or diet is determined using a polyphenol diversity index.
4. The method according to any preceding para, wherein a greater sum of different types of polyphenol present in the food product, meal, or diet provides a more diverse gut microbiota.
5. A method for determining the effects of a food product, meal, or diet on a subject's gut microbiota, wherein the method comprises determining the diversity of polyphenols present in a food product, meal, or diet, preferably wherein the diversity of polyphenols present in the food product, meal, or diet is determined using a polyphenol diversity index.
6. The method according to any of paras 3 to 5, wherein a greater diversity of polyphenols present in the food product, meal, or diet provides a more diverse gut microbiota.
7. The method according to para 4 or 6, wherein a more diverse gut microbiota has:
   (i) a higher number of observed microbial taxa; and/or
   (ii) a higher diversity index, preferably wherein the diversity index is a phylogenetic diversity index, such as a Faith's PD index, or a Shannon index.
8. The method according to any preceding para, wherein the different types of polyphenol comprise flavonoids, lignans, stilbenes, and phenolic acids.
9. The method according to any preceding para, wherein the different types of polyphenol comprise flavonols, flavanones, flavones, flavan-3-ols, anthocyanins/anthocyanidins, and isoflavones.
10. The method according to any preceding para, wherein the different types of polyphenol comprise flavonols, flavanones, flavones, catechins, theaflavins, thearubigins, anthocyanins/anthocyanidins, and isoflavones.
11. The method according to any preceding para, wherein the different types of polyphenol comprise: (−)-Epicatechin, (−)-Epicatechin 3-gallate, (−)-Epigallocatechin, (−)-Epigallocatechin 3-gallate, (+)-Catechin, (+)-Gallocatechin, Apigenin, Cyanidin, Daidzein, Delphinidin, Eriodictyol, Genistein, Glycitein, Hesperetin, Isorhamnetin, Kaempferol, Luteolin, Malvidin, Myricetin, Naringenin, Pelargonidin, Peonidin, Petunidin, Quercetin, Theaflavin, Theaflavin-3-gallate, Theaflavin-3,3′-digallate, Theaflavin-3′-gallate, and Total Thearubigins.
12. The method according to any preceding para, wherein the different types of polyphenol comprise: (−)-Epicatechin, (−)-Epicatechin 3-gallate, (−)-Epigallocatechin, (−)-Epigallocatechin 3-gallate, (+)-Catechin, (+)-Gallocatechin, Apigenin, Cyanidin, Daidzein, Delphinidin, Eriodictyol, Genistein, Glycitein, Hesperetin, Isorhamnetin, Kaempferol, Luteolin, Malvidin, Myricetin, Naringenin, Pelargonidin, Peonidin, Petunidin, Quercetin, Theaflavin, Theaflavin-3-gallate, Theaflavin-3,3′-digallate, Theaflavin-3′-gallate, Total Thearubigins, Total anthocyanidins, Total catechins, Total flavan-3-ols, Total flavanones, Total flavones, Total flavonoids, Total flavonols, and Total isoflavones.
13. The method according to any preceding para, wherein the method comprises determining a dietary index using the sum of different types of polyphenol present in the food product, meal, or diet.
14. The method according to any preceding para, wherein the method comprises determining a dietary index using the amount of each different type of polyphenol present in the food product, meal, or diet and/or the similarity in the chemical structure of the different types of polyphenol present in the food product, meal, or diet.
15. The method according to any preceding para, wherein the method comprises determining a dietary index using the diversity of polyphenols present in the food product, meal, or diet, preferably wherein the diversity of polyphenols present in the food product, meal, or diet is determined using a polyphenol diversity index.
16. The method according to any of paras 13 to 15, wherein a higher dietary index provides a more diverse gut microbiota.
17. A dietary index for assessing the effects of a food product, meal, or diet on a subject's gut microbiota, wherein the dietary index is determined using the sum of different types of polyphenol present in the food product, meal, or diet.
18. The dietary index according to para 17, wherein the dietary index comprises or consists of the sum of different types of polyphenol present in the food product, meal, or diet.
19. The dietary index according to para 17 or 18, wherein the amount of each different type of polyphenol present in the food product, meal, or diet and/or the similarity in the chemical structure of the different types of polyphenol present in the food product, meal, or diet is also used to determine the dietary index.
20 The dietary index according to any of paras 16 to 19, wherein the dietary index is determined using the diversity of polyphenols present in the food product, meal, or diet, preferably wherein the diversity of polyphenols present in the food product, meal, or diet is determined using a polyphenol diversity index.
21. The dietary index according to any of paras 16 to 20, wherein the dietary index comprises or consists of a polyphenol diversity index.
22. The dietary index according to any of paras 16 to 21, wherein the different types of polyphenol comprise flavonoids, lignans, stilbenes, and phenolic acids.
23. The dietary index according to any of paras 16 to 22, wherein the different types of polyphenol comprise flavonols, flavanones, flavones, flavan-3-ols, anthocyanins/anthocyanidins, and isoflavones.
24. The dietary index according to any of paras 16 to 23, wherein the different types of polyphenol comprise flavonols, flavanones, flavones, catechins, theaflavins, thearubigins, anthocyanins/anthocyanidins, and isoflavones.
25. The dietary index according to any of paras 16 to 24, wherein the different types of polyphenol comprise: (−)-Epicatechin, (−)-Epicatechin 3-gallate, (−)-Epigallocatechin, (−)-Epigallocatechin 3-gallate, (+)-Catechin, (+)-Gallocatechin, Apigenin, Cyanidin, Daidzein, Delphinidin, Eriodictyol, Genistein, Glycitein, Hesperetin, Isorhamnetin, Kaempferol, Luteolin, Malvidin, Myricetin, Naringenin, Pelargonidin, Peonidin, Petunidin, Quercetin, Theaflavin, Theaflavin-3-gallate, Theaflavin-3,3′-digallate, Theaflavin-3′-gallate, and Total Thearubigins.
26. The dietary index according to any of paras 16 to 25, wherein the different types of polyphenol comprise: (−)-Epicatechin, (−)-Epicatechin 3-gallate, (−)-Epigallocatechin, (−)-Epigallocatechin 3-gallate, (+)-Catechin, (+)-Gallocatechin, Apigenin, Cyanidin, Daidzein, Delphinidin, Eriodictyol, Genistein, Glycitein, Hesperetin, Isorhamnetin, Kaempferol, Luteolin, Malvidin, Myricetin, Naringenin, Pelargonidin, Peonidin, Petunidin, Quercetin, Theaflavin, Theaflavin-3-gallate, Theaflavin-3,3′-digallate, Theaflavin-3′-gallate, Total Thearubigins, Total anthocyanidins, Total catechins, Total flavan-3-ols, Total flavanones, Total flavones, Total flavonoids, Total flavonols, and Total isoflavones.
27. The dietary index according to any of paras 16 to 26, wherein a higher dietary index provides a more diverse gut microbiota.
28. The dietary index according to para 27, wherein a more diverse gut microbiota has:
   (i) a higher number of observed microbial taxa; and/or
   (ii) a higher diversity index, preferably wherein the diversity index is a phylogenetic diversity index, such as a Faith's PD index, or a Shannon index.
29. A method for providing a dietary index according to any of paras 16 to 28, wherein the method comprises determining the dietary index using the sum of different types of polyphenol present in a food product, meal, or diet.
30. A method for adjusting a food product, meal, or diet, wherein the method comprises:
   (a) determining the effects of the food product, meal, or diet on a subject's gut microbiota using a method according to any of paras 1 to 15; and
   (b) adjusting the food product, meal, or diet to provide an adjusted food product, adjusted meal, or adjusted diet with an improved effect on the subject's gut microbiota diversity.
31. The method according to para 30, wherein the adjusted food product, adjusted meal, or adjusted diet provides a greater number of different types of polyphenol than the un-adjusted food product, un-adjusted meal, or un-adjusted diet.
32. A method for maintaining or improving a subject's gut microbiota diversity, wherein the method comprises:
   (a) determining the effects of the subject's present diet on the subject's gut microbiota using a method according to any of paras 1 to 15; and
   (b) adjusting the subject's present diet to provide an adjusted diet with an improved effect on the subject's gut microbiota diversity.
33. A method for maintaining or improving a subject's gut microbiota diversity, wherein the method comprises:
   (a) determining a dietary index according to any of paras 16 to 28 for the subject's present diet; and
   (b) adjusting the subject's present diet to provide an adjusted diet with an improved dietary index.
34. The method according to para 32 or 33, wherein the adjusted diet provides a greater number of different types of polyphenol than the subject's present diet.
35. Use of the sum of different types of polyphenol present in a food product, meal, or diet to assess the effects of the food product, meal, or diet on a subject's gut microbiota.
36. Use of a polyphenol diversity index to assess the effects of the food product, meal, or diet on a subject's gut microbiota.
37. Use of a dietary index according to any of paras 16 to 28 for assessing the effects of a food product, meal, or diet on a subject's gut microbiota.
38. A computer program comprising instructions which, when the program is executed by a computer, cause the computer to carry out a method according to any of paras 1 to 15 or 29 to 34.
39. A computer program comprising instructions which, when the program is executed by a computer, cause the computer to determine a dietary index for assessing the effects of a food product, meal, or diet on a subject's gut microbiota, given the different types of polyphenol present in the food product, meal, or diet.
40. A computer-readable medium comprising instructions which, when executed by a computer, cause the computer to carry out a method according to any of paras 1 to 15 or 29 to 34.
41. A computer-readable medium comprising instructions which, when executed by a computer, cause the computer to determine a dietary index for assessing the effects of a food product, meal, or diet on a subject's gut microbiota, given the different types of polyphenol present in the food product, meal, or diet.

All publications mentioned in the above specification are herein incorporated by reference. Various modifications and variations of the disclosed methods, uses, computer-implemented methods and indices of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A method for determining the effects of a food product, meal, or diet on a subject's gut microbiota, wherein the method comprises determining the sum of different types of polyphenol present in the food product, meal, or diet.

2. The method according to claim 1, wherein a greater sum of different types of polyphenol present in the food product, meal, or diet provides a more diverse gut microbiota.

3. The method according to claim 1, wherein the different types of polyphenol are selected from the group consisting of flavonoids, lignans, stilbenes, and phenolic acids.

4-7. (canceled)

8. A method for adjusting a food product, meal, or diet, wherein the method comprises:
(a) determining the effects of the food product, meal, or diet on a subject's gut microbiota using a method comprising determining the sum of different types of polyphenol present in the food product, meal, or diet; and
(b) adjusting the food product, meal, or diet to provide an adjusted food product, adjusted meal, or adjusted diet with an improved effect on the subject's gut microbiota diversity.

9. A method for maintaining or improving a subject's gut microbiota diversity, wherein the method comprises:
(a) determining the effects of the subject's present diet on the subject's gut microbiota using a method for determining the effects of a food product, meal, or diet on a subject's gut microbiota, wherein the method comprises determining the sum of different types of polyphenol present in the food product, meal, or diet; and
(b) adjusting the subject's present diet to provide an adjusted diet with an improved effect on the subject's gut microbiota diversity.

10-15. (canceled)
